(19) United States
(12) Patent Application Publication
(10) Pub. No.: US 2009/0246156 A1
(43) Pub. Date: Oct. 1, 2009

(54) ANTI-ACNE SUNSCREEN COMPOSITION

(76) Inventor: Audrey Kunin, Mission Hills, KS (US)

Correspondence Address: HUSCH BLACKWELL SANDERS LLP, 4801 Main Street, Suite 1000, KANSAS CITY, MO, 64112, US

(21) Appl. No.: 12/059,761

(22) Filed: Mar. 31, 2008

(51) Int. Cl. A61K 8/33; A61K 8/30; A61Q 17/04

(52) U.S. Cl. 424/60; 424/59

(57) ABSTRACT

The present invention is directed to compositions for anti-acne sunscreen. The sunscreen composition has the unique ability to treat and prevent acne in addition to screen both UVA and UVB radiation. In particular, the sunscreen composition includes a sunscreen base, at least one UVA deactivator, at least one UVB deactivator, and at least one anti-acne agent. The UVA deactivator may be avobenzone and the UVB deactivator may be selected from one of the following oxybenzone, octisalate, octyl methoxycinnamate, or a mixture thereof.

### CROSS-REFERENCE TO RELATED APPLICATIONS

None.

### BACKGROUND OF THE INVENTION

The present invention generally relates to compositions and methods for producing anti-acne sunscreen. Acne vulgaris, or acne, is a common skin disease is prevalent in teenagers and young adults. Acne is associated with low self-esteem and social inhibition in those that suffer from acne especially if it is particularly severe. Several factors may contribute to the development of acne. The primary problem is that the abnormal flaking of cells inside the hair follicle leads to the formation of a plug. The plug can enlarge and even rupture the hair follicle. A ruptured hair follicle spills its contents of oil and debris into the skin where it leads to swelling and causes redness (inflammation). Natural occurring bacteria known as Propionibacterium acnes may also be responsible for causing acne. These bacteria produce substances that cause redness and irritation (inflammation) and also make enzymes, which dissolve the sebum (oil from oil glands in the skin) into irritating substances. These substances also make the inflammation worse.

Certain hormones called androgens can be an additional factor in causing acne. Androgens are male hormones that are present in both men and women and do two things (1) enlarge the sebaceous glands in the skin, and (2) cause glands to increase sebum production. The increased sebum leads to plug formation and serves as more “food” for the bacteria. Androgens surge at puberty, which contributes to the development of acne in teenagers and young adults.

Hair follicles exist on virtually all skin except for the palms of the hands and soles of the feet. Inside the follicle, the hair extends up from the deep layers of the skin and comes out of a pore. Near the surface, the oil gland (sebaceous gland) enters the hair follicle where it empties sebum at a relatively constant rate. The sebum lubricates the skin and provides a protective barrier to prevent drying. Skin on the face, chest, and back has an especially large number of sebaceous glands. These are the areas where acne is most likely to occur.

There are two major types of acne lesions noninflammatory and inflammatory. Noninflammatory acne lesions include blackheads (open comedones) and whiteheads (closed comedones). Open and closed comedones along with papules and pustules are referred to as papulopustular acne—a form of inflammatory acne. Nodular acne is the most severe form of inflammatory acne. Open comedones result from the enlargement and dilation of a plug that forms from oil and flakes of skin inside the hair follicle. A closed comedo forms if the hair follicle pore remains closed. Inflammatory acne lesions consist of red blemishes, pimples also called zits (papules, pustules), and larger, deeper swollen tender lesions (nodules). Papules are closed comedos, which have become red, swollen, and inflamed. Pustules are closed comedos, which become inflamed and begin to rupture into the skin forming pustular heads of various sizes. Nodules represent large, tender, swollen acne lesions, which have become intensely inflamed and rupture under the skin. If untreated, these can produce deep scarring.

There are many different topical therapies that are available to treat and prevent acne. Topical retinoids such as tretinoin or adapalene are effective for treating comedonal acne. Inflammatory lesions benefit from treatment with benzoyl peroxide, azelaic acid or topical antibiotics. People that are prone to acne have to be very careful what type of lotions and sunscreens that they use on their faces and torso so as to not make their acne worse. Most over-the-counter sunscreens are loaded with oils that may clog pores and add to the cycle of acne. Therefore, it would be beneficial to have a sunscreen composition that aids in treating and preventing acne rather than contributing to it.

Sunscreens are important in preventing skin cancer and actinic keratoses, which are warty lesions that can occur on sun exposed skin of the face or hands. Research has shown that these lesions can develop into a cancer called squamous cell carcinoma, and that this is linked to a cumulative exposure to the sun. UVA rays have longer wavelengths and are recognized as a deep-penetrating radiation. Long-term exposure can damage the skin's connective tissues, leading to premature aging and playing a role in the development of skin cancer. UVB rays have shorter wavelengths and are primarily responsible for sunburn and also may contribute to skin cancer. Both UVA and UVB radiation have been linked to skin cancer. Therefore, it would be beneficial to have a sunscreen composition that screens both UVA and UVB radiation. UVA radiation may also contribute to cutaneous aging, immunosuppression, polymorphous light eruption and urticaria. Therefore, it would be beneficial to have a sunscreen composition that aids in protecting against UVA rays.

### SUMMARY OF THE INVENTION

The present invention is directed to compositions for anti-acne sunscreen. The sunscreen composition has the unique ability to treat and prevent acne in addition to screen both UVA and UVB radiation. In particular, a sunscreen composition includes a sunscreen base, at least one UVA deactivator, at least one UVB deactivator, and at least one anti-acne agent. In one noon limiting illustration the sunscreen composition contains avobenzone, oxybenzone, octisalate, octyl methoxycinnamate, and at least one an anti-acne agent.

Other and further objects of the invention, together with the features of novelty appurtenant thereto, will appear in the course of the following description.

### DETAILED DESCRIPTION OF THE INVENTION

There is provided herein a sunscreen composition that prevents and treats acne while also blocking harmful UVA and UVB rays. The sunscreen composition generally includes a sunscreen base, at least one UVA deactivator, at least one UVB deactivator, and an anti-acne agent. Preventing skin cancer by using sunscreen is already known in the industry. Due to the composition of some sunscreens, some types of sunscreen may aid in the development of acne. The novel combination of a sunscreen that blocks both UVA and UVB radiation and treats and prevents acne has resulted in a unique sunscreen composition.

In one embodiment, the sunscreen base has at least one UVA deactivator and at least one UVB deactivator. The sunscreen base may be but is not limited to soaps, shampoos, washes, lotions, creams, gels, masks, ointments, solutions, scrubs, microdermabrasion creams, serums, strips, and patches. In one embodiment, the UVA deactivator may be but is not limited to avaobenzone. In another embodiment, the UVB deactivator may be but is not limited to oxybenzone, oxtisalate, octyl methoxycinnamate or a mixture thereof.

In one embodiment, a sunscreen composition includes at least one UVA deactivator, at least one UVB deactivator, at least one anti-acne agent, advanced polymer technology, botanically derived agents, and natural botanicals. The UVA deactivator may be avaobenzone and is preferably about 3%. The UVB deactivator may be oxybenzone and is preferably about 6%. Another UVB deactivator may be octisalate and is preferably about 5%. Yet another UVB deactivator may be octyl methoxycinnamate and is preferably about 7.5%. In one embodiment, the UVB deactivator is a mixture of 6% oxybenzone, 5% octisalate, and 7.5% octyl methoxycinnamate. In one embodiment, the anti-acne agent may be salix alba bark extract (willow bark), hamamelis Virginiana distillate (witch hazel), or a mixture thereof. Willow bark is a natural source of salicyclic acid thought to clarify blemish-prone, oily or combination skin. Witch hazel is a natural astringent that gently leaves the complexion radiant. The advanced polymer technology soaks up excess oils and mattifies without leaving skin parched that aids in prevention of acne. The botanically derived agents help calm and sooth inflamed and irritated skin. The natural botanicals are an ideal antidote for hormonally out-of-control skin. In one embodiment, the sunscreen composition includes an anti-inflammatory agent. In one embodiment, sunscreen composition may include an anti-bacterial agent such as bacitracin, polymyxin B, neomycin, mupirocin, or any other anti-bacterial that is now known or that may be known in the future.

In one embodiment, the sunscreen composition includes about 3% avobenzone, about 7.5% octyl methoxycinnamate, about 6% oxybenzone, about 5% octisalate, deionized water, C-12-C-15 alkyl benzoate, salix alba bark extract, butylenes glycol, PEG-60 almond glycerides, caprylyl glycol, nordihydroguaiaretic acid, oleanolic acid, glycerine, butyloctyl salicylate, cyclomethicone, glycyrrhiza, glabra root extract, methyl methacrylate/glycol dimethacrylate crosspolymer, sodium hyaluronate, hamamelis virginiana distillate, acrylate/C 10-30 alkyl acrylate crosspolymer, carbomer, TEA carbomer, tocopheryl acetate, disodium EDTA, sorbitan oleate, propylene glycol, cucumis sativus (cucumber) fruit extract, arnica Montana flower extract, anacyclus pyrethrum root extract, phenoxyethanol, methylparaben, ethylparaben, propylparaben, butylparaben, and mixtures thereof.

Nordihydroguaiaretic acid (NDGA) has important anti-inflammatory, anti-acne, and anti-aging properties, blocks UV effects, and blocks matrix degradation. NDGA mitigates the inflammatory response in skin. NDGA inhibits the synthesis of inflammatory mediators such as prostaglandins and leukotrines by blocking the action of the enzyme lipoxygenase. NDGA is able to block chemically induced skin irritation. NDGA indirectly inhibits the production of enzymes that can break down the skin and reduces fine lines and wrinkles. NDGA also reduces the amount of sebum available to nourish bacteria or plug pores and helps clear visible blemishes. NDGA is a bi-phenolic compound that is a component of the resinous exudates of many plants. In its structure, NDGA has two moles of antioxidant power in one molecule. Traditionally extracted from a plant (larrea divaricata), NDGA has been used for years as an oil-soluble antioxidant.

Glycyrrhiza glabra (licorice) root extract has main constituents of glycyrrhizin, potassium and calcium salts of glycyrrhitinic acid. Glycyrrhiza glabra has shown to have anti-inflammatory effects.

Other suitable anti-acne agents for use in the present invention include, but are not limited to, benzoyl peroxide, benzoyl peroxide and clindamycin combinations, clindamycin, erythromycin, tetracycline, alpha hydroxyl acid, salicylic acid, filipendula and meadowsweet, curled dock (rumex crispus), evodia rutaecarpa, boswellia serrata, licorice extract, quercetin, zinc sulphate, alum, achillea and yarrow combinations, minocycline, doxycycline, trimethoprim, trimethoprim and sulfacetamide combinations, zinc oxide, pyrithione zinc, linoleic acid, azithromycin, lymecycline, ethinyl estradiol, adapalene-benzoyl peroxide, drospirenone, ethinyl estradiol, solumedrol, azathiprine, imiquimod, tacrolimus, sirolimus, alpha binding proteins such as infliximab, etanercept or adalimumab, cyclosporin, myriocin, fingolimod, methotrexate, cox-2 inhibitors, acetic acid, acetaminophen, acetylsalicyclic acid, prednisone, prednisolone, cortisone, hydrocortisone, microdermabrasion creams, glycolic acid, tea tree oil, witch hazel, eucalyptus oil, tretinoin, tretinoin microsphere, metronidazole, adapalene, sulfacetamide, sulfur and sulfacetamide combinations, sulfur and sulfacetamide and urea combinations, resourcinol, sulfur and resourcinol combinations, azeleic acid, isotrentinoin, dapsone, atrisone, salicyclic acid, ketoconazole, miconazole, doxycycline, DHT inhibitors such as spironolactone, drosperinone, propecia, bcps, and nordihydroguaiaretic acid, flutamide, cyproterone acetate, finasteride, aminolevulinic acid HCL, derivatives thereof, and mixtures thereof.

In an alternative embodiment, the sunscreen base is green tea extract. The green tea extract acts as both a UVA deactivator and a UVB deactivator. Green tea extraction may reduce the risk of cancer development and protects against both UVA and UVB rays.

From the foregoing it will be seen that this invention is one well adapted to attain all ends and objects hereinabove set forth together with the other advantages which are obvious and which are inherent to the structure.

It will be understood that certain features and subcombinations are of utility and may be employed without reference to other features and subcombinations. This is contemplated by and is within the scope of the claims.

Since many possible embodiments may be made of the invention without departing from the scope thereof, it is to be understood that all matter herein set forth or shown in the accompanying drawings is to be interpreted as illustrative, and not in a limiting sense.

What is claimed is:

1. A sunscreen composition comprising:
a sunscreen base
at least one UVA deactivator;
at least one UVB deactivator; and
at least one anti-acne agent.

2. The composition of claim 1 wherein said sunscreen base is selected from the group consisting of soaps, shampoos, washes, lotions, creams, gels, masks, ointments, solutions, scrubs, microdermabrasion creams, serums, strips, and patches.

3. The composition of claim 1 wherein said UVA deactivator is avobenzone.

4. The composition of claim 1 wherein said UVB deactivator is selected from a group consisting of oxybenzone, octisalate, octyl methoxycinnamate, and mixtures thereof.

5. The composition of claim 1 wherein said UVA deactivator is about 3% avobenzone, and wherein said UVB deactivator is selected from a group consisting of about 6% oxybenzone, about 5% octisalate, and about 7.5% octyl methoxycinnamate.

6. The composition of claim 1 wherein said anti-acne agent is selected from the group consisting of benzoyl peroxide, clindamycin, erythromycin, tetracycline, alpha hydroxyl acid, salicylic acid, filipendula, meadowsweet, curled dock, evodia rutaccarpa, boswellia serrata, licorice extract, quercetin, zinc sulphate, alum, achillea, yarrow, minocycline, doxycycline, trimethoprim, sulfacetamide, zinc oxide, pyrithione zinc, linoleic acid, azithromycin, lymecycline, ethinyl estradiol, adapalene-benzoyl peroxide, drospirenone, ethinyl estradiol, solumedrol, azathiprine, imiquimod, tacrolimus, sirolimus, cyclosporine, myriocin, fingolimod, methotrexate, cox-2 inhibitors, acetic acid, acetaminophen, acetylsalicyclic acid, prednisone, prednisolone, cortisone, hydrocortisone, glycolic acid, tea tree oil, alpha binding proteins, witch hazel, eucalyptus oil, tretinoin, tretinoin microsphere, metronidazole, adapalene, sulfacetamide, sulfur, urea, resourcinol, azeleic acid, isotrentinoin, dapsone, atrisone, salicyclic acid, ketoconazole, miconazole, doxycycline, DHT inhibitors, flutamide, cyproterone acetate, finasteride, aminolevulinic acid HCL, derivatives thereof, and mixtures thereof.

7. The composition of claim 6 wherein said DHT inhibitors are selected from the group consisting of spironolactone, drosperinone, propecia, bcps, nordihydroguaiaretic acid and mixtures thereof.

8. The composition of claim 6 wherein said alpha binding proteins are selected from the group consisting of infliximab, etanercept, adalimumab, and mixtures thereof.

9. The composition of claim 1 comprising an anti-inflammatory agent

10. The composition of claim 1 comprising an anti-bacterial agent.

11. A sunscreen composition comprising:
a sunscreen base, wherein said sunscreen base is selected from a group consisting of soaps, shampoos, washes, lotions, creams, gels, masks, ointments, solutions, scrubs, microdermabrasion creams, serums, strips, and patches;
at least one UVA deactivator, wherein said UVA deactivator is avobenzone;
at least one UVB deactivator, wherein said UVB deactivator is selected from a group consisting of octyl methoxycinnamate, oxybenzone, octisalate, and mixtures thereof;
at least one anti-acne agent, wherein said anti-acne agent is selected from a group consisting of salix alba bark extract, hamamelis Virginiana distillate, benzoyl peroxide, clindamycin, erythromycin, tetracycline, alpha hydroxyl acid, salicylic acid, filipendula, meadowsweet, curled dock, evodia rutaecarpa, boswellia serrata, licorice extract, quercetin, zinc sulphate, alum, achillea, yarrow, minocycline, doxycycline, trimethoprim, sulfacetamide, zinc oxide, pyrithione zinc, linoleic acid, azithromycin, lymecycline, ethinyl estradiol, adapalene-benzoyl peroxide, drospirenone, ethinyl estradiol, solumedrol, azathiprine, imiquimod, tacrolimus, sirolimus, cyclosporine, myriocin, fingolimod, methotrexate, cox-2 inhibitors, acetic acid, acetaminophen, acetylsalicyclic acid, prednisone, prednisolone, cortisone, hydrocortisone, glycolic acid, tea tree oil, alpha binding proteins, witch hazel, eucalyptus oil, tretinoin, tretinoin microsphere, metronidazole, adapalene, sulfacetamide, sulfur, urea, resourcinol, azeleic acid, isotrentinoin, dapsone, atrisone, salicyclic acid, ketoconazole, miconazole, doxycycline, DHT inhibitors, flutamide, cyproterone acetate, finasteride, aminolevulinic acid HCL, and mixtures thereof; and
at least one additive, wherein said additive is selected from a group consisting of deionized water, C-12-C-15 alkyl benzoate, butylenes glycol, PEG-60 almond glycerides, caprylyl glycol, nordihydroguaiaretic acid, oleanolic acid, glycerine, butyloctyl salicylate, cyclomethicone, glycyrrhiza glabra root extract, methyl methacrylate/glycol dimethacrylate crosspolymer, sodium hyaluronate, acrylate/C10-30 alkyl acrylate crosspolymer, carbomer, TEA carbomer, tocopheryl acetate, disodium EDTA, sorbitan oleate, propylene glycol, cucumis sativus fruit extract, arnica Montana flower extract, anacyclus pyrethrum root extract, phenoxyethanol, methylparaben, ethylparaben, propylparaben, butylparaben, and mixtures thereof.

12. The composition of claim 11 wherein said avobenzone is about 3%.

13. The composition of claim 11 wherein said oxybenzone is about 6%.

14. The composition of claim 11 wherein said octisalate is about 5%.

15. The composition of claim 11 wherein said octyl methoxycinnamate is about 7.5%.

16. A sunscreen composition comprising:
a sunscreen base, wherein said sunscreen base is selected from a group consisting of soaps, shampoos, washes, lotions, creams, gels, masks, ointments, solutions, scrubs, microdermabrasion creams, serums, strips, and patches;
at least one UVA deactivator, wherein said UVA deactivator is about 3% avobenzone;
at least one UVB deactivator, wherein said UVB deactivator is selected from a group consisting of about 6% octyl methoxycinnamate, about 5% oxybenzone, about 7.5% octisalate, and mixtures thereof;
at least one anti-acne agent, wherein said anti-acne agent is selected from a group consisting of salix alba bark extract, hamamelis Virginiana distillate, and mixtures thereof; and
at least one additive, wherein said additive is selected from a group consisting of deionized water, C-12-C-15 alkyl benzoate, butylenes glycol, PEG-60 almond glycerides, caprylyl glycol, nordihydroguaiaretic acid, oleanolic acid, glycerin, butyloctyl salicylate, cyclomethicone, glycyrrhiza glabra root extract, methyl methacrylate/glycol dimethacrylate crosspolymer, sodium hyaluronate, acrylate/C10-30 alkyl acrylate crosspolymer, carbomer, TEA carbomer, tocopheryl acetate, disodium EDTA, sorbitan oleate, propylene glycol, cucumis sativus fruit extract, arnica Montana flower extract, anacyclus pyrethrum root extract, phenoxyethanol, methylparaben, ethylparaben, propylparaben, butylparaben, and mixtures thereof.
